(19) United States
(12) Patent Application Publication
(10) Pub. No.: US 2022/0064592 A1
(43) Pub. Date: Mar. 3, 2022

(54) CELL MICROSHEET, SYRINGE CONTAINING THE CELL MICROSHEET, AND PRODUCTION AND USE OF THE CELL MICROSHEET

(71) Applicants: TOKAI UNIVERSITY EDUCATIONAL SYSTEM, Tokyo (JP); CellSeed Inc., Tokyo (JP)

(72) Inventors: Masato SATO, Isehara-shi (JP); Shiho WASAI, Isehara-shi (JP); Yuka KAWAGUCHI, Tokyo (JP)

(73) Assignees: TOKAI UNIVERSITY EDUCATIONAL SYSTEM, Tokyo (JP); CellSeed Inc., Tokyo (JP)

(21) Appl. No.: 17/465,425

(22) Filed: Sep. 2, 2021

(30) Foreign Application Priority Data
Sep. 3, 2020 (JP) 2020-148200

(51) Int. Cl. C12N 5/00; A61K 9/00; C12N 5/077; A61K 35/32; A61L 27/54; A61L 27/36

(52) U.S. Cl. CPC C12N 5/0075; A61K 9/0019; C12N 5/0655; A61K 35/32; A61L 27/54; A61L 2300/412; A61L 27/3612; A61L 2430/06; A61L 2400/06; C12N 2531/00; A61L 2300/64; A61L 27/3654

(57) ABSTRACT

Cell microsheets are formed from a culture of cells. The cell microsheets has a size that can pass through an injection needle with a certain thickness. The cell microsheets can be produced on a surface of a cell cultureware. A stimulus-responsive polymer is immobilized on the surface having small divisions of the cell cultureware. The cell microsheets are suitable for minimally invasive treatment.

### TECHNICAL FIELD

The present invention relates to a cell microsheet, a syringe containing the cell microsheet, and production and use of the cell microsheet.

### BACKGROUND ART

For the treatment of locomotor disorders, such as osteoarthritis, cartilage tissue is being treated by tissue regeneration engineering technology. This treatment involves transplantation of cultured chondrocytes or cartilage tissues made from chondrocytes into the affected area. Various transplant materials have been proposed.

For example, Japanese Unexamined Patent Application Publication No. 2003-180819 discloses, in claim 1, “a material for transplantation to be transplanted to a predetermined transplantation site. In the material for transplantation, cells corresponding to a predetermined transplantation site are retained on a cell retention carrier that is obtained by subjecting a tissue structure of the same type as the predetermined transplantation site obtained from a body tissue to an antigenicity suppression treatment while maintaining the shape of the tissue structure.”

### CITATION LIST

Japanese Unexamined Patent Application Publication No. 2003-180819

### SUMMARY OF INVENTION

### Technical Problem

Treatment using transplant materials sometimes requires a highly invasive method in which the affected area is exposed by incision before transplantation under direct vision.

The main object of the present invention is to provide a cell microsheet suitable for minimally invasive treatment.

### Solution to Problem

The present invention provides a cell microsheet that is formed from a culture of cells and is capable of passing through an injection needle.

The injection needle may be an 18G or thinner injection needle.

The cell microsheet may have an area of 20 mm² or less.

The cell microsheet may be usable for cartilage tissue repair.

The cells may be derived from cartilage tissue.

The cartilage tissue may be of an animal with polydactyly.

The cells may be derived from stem cells.

The stem cells may include pluripotent stem cells, embryonic stem cells, or somatic stem cells.

The stem cells may include iPS cells.

The present invention also provides a syringe containing the cell microsheet.

The present invention further provides a method of producing cell microsheets formed from a culture of cells, comprising cultivating the cells on a surface of a cell cultureware to yield the cell microsheets, a stimulus-responsive polymer being immobilized on the surface of the cell cultureware, the surface having small divisions.

The small divisions may each have an area of 20 mm² or less.

The present invention still further provides a method of administering the cell microsheets to an animal by injection.

### Advantageous Effect

The present invention provides a cell microsheet suitable for minimally invasive treatment.

### BRIEF DESCRIPTION OF DRAWINGS

FIG. 1 is a graph illustrating the experimental results on number of cells and viability of cells.

FIG. 2 is a graph illustrating the experimental results on secretion of humoral factor.

FIG. 3 is a graph illustrating the results of quantitative analysis of cartilage-related gene expression.

FIG. 4 is a graph illustrating the analytical results of the cell surface marker.

FIG. 5 is a photograph illustrating the results of evaluation of tissue sections.

FIG. 6 is a graph illustrating the experimental results of viability of cells after injection administration.

### EMBODIMENTS OF INVENTION

Section 1. Cell microsheet

The cell microsheets of the present invention can passes through injection needles. The passage of the cell microsheets through the injection needle can be confirmed with a solution containing the cell microsheets. In detail, a syringe provided with an injection needle and a plunger is filled with a solution containing the cell microsheets and then is pressed to confirm whether the cell microsheet can be ejected from the injection needle. The cell microsheet ejected from the injection needle can be regarded as one that can pass though the injection needle.

The solution can be appropriately prepared by persons skilled in the art. Preferably, the solution can be prepared to increase the viability of cells of the cell microsheets in the solution. The viability of cells of the cell microsheets in the solution is preferably 80% or more. In other words, the viability of cells in the cell microsheets of the present invention before cells pass through the injection needle is preferably 80% or more. The viability of cells in the cell microsheets of the present invention may be determined by trypan blue assay.

The cell microsheet of the present invention, which can pass through injection needles, can be used for treatment by injection. Treatment with a conventional cell sheet involves exposure of an affected area by a surgical procedure and application of the cell sheet onto the exposed affected area in many cases. Such conventional treatment is highly invasive and puts a heavy burden on a patient. In contrast, treatment by administration by injection of the cell microsheets of the present invention is lowly invasive compared with conventional treatment and reduce the burden on the patient. In conclusion, the cell microsheet of the present invention is suitable for minimally invasive treatment.

The injection needle should preferably be an 18G or thinner injection needle. In other words, the injection needle should preferably have an outer diameter of 1.2 mm or less. In the present invention, cell microsheets are more susceptible while they pass through a thinner injection needle. It is thus preferred that the injection needle has a thickness of 23G or more. In other words, the injection needle should preferably have an outer diameter of 0.6 mm or more.

It is preferred that the viability of cells of the cell microsheets of the present invention immediately after passing through the injection needle does not significantly decrease from the viability of cells before passing through the injection needle. The difference in viability of cells between before and immediately after passing through the injection needle may be, for example, 10 points or less. In the cell microsheet of the present invention, the difference in viability of cells between before passing through the injection needle and after 24 hours after passing through the injection needle may be, for example, 10 points or less. A smaller difference in viability of cells before and after passing through the injection needle indicates that the cell microsheets of the present invention are less susceptible to injection. Such cell microsheets are more suitable for administration by injection.

The cell microsheet of the present invention should preferably have a viability of cells of 80% or more immediately after passing through the injection needle. Furthermore, the cell microsheet of the present invention should preferably have a viability of cells of 80% or more after 24 hours after passing through the injection needle.

The cell microsheet of the present invention is a small piece of sheet that has a two-dimensionally spreading area and a thickness considerably smaller than the area.

Cell microsheets having smaller areas can readily pass through injection needles. Accordingly, the cell microsheet of the present invention has an area of preferably 20 mm² or less, more preferably 15 mm² or less, most preferably 10 mm² or less. An excessively smaller area may, however, preclude formation of cell microsheets from a culture of cells. Accordingly the area should preferably be 0.02 mm² or more.

The cell microsheets of the present invention can be formed through cultivation of cells on a cell cultureware and recovered through detachment of the cells from the cell cultureware. The cell microsheets may sometimes be shrinkable. In such a case, the cell microsheets will have different apparent areas between a state present on the cell cultureware and a state after detachment from the cell cultureware. In the case of shrinkable cell microsheets, the microsheets are detached from the cell cultureware and then the detached cell microsheets are placed onto a flat face to measure their areas.

In some cases, the shrinkable cell microsheets may be shrunk into a mass after detachment from the cell cultureware. Even in such a case, the shape of the cell microsheets of the present invention can be confirmed after the shrunk cell microsheets are spread onto a flat surface. For example, spheroids formed by three-dimensional cultivation of cells are massive forms not sheet forms; hence, the spheroids are not categorized into the cell microsheet of the present invention. Accordingly, the cell microsheet of the present invention does not include spheroid.

In one embodiment of the present invention, the cell microsheet may preferably be used for cartilage tissue repair. Conventional cell sheets for cartilage tissue repair have often been applied to affected sites by highly invasive surgical procedures. In contrast, the cell microsheets for cartilage tissue repair of the present invention can be applied by injection; hence, the cell microsheets can be effective for lowly invasive treatment.

Examples of the cartilage tissue repair in the present invention include, but not limited to, treatment of inflammatory and/or damaged cartilage tissues, reinforcement of cartilage tissues, prosthesis of cartilage tissue defects, and regeneration of cartilage tissues. The cell microsheets of the present invention can also be used in disease prevention on cartilage tissue. The cell microsheets of the present invention can be applied by injection into diseased cartilage or bone tissues. Examples of disease to which the cell microsheets of the present invention is applicable include, but not limited to, arthritis, arthropathy, cartilage injury, osteochondral injury, meniscus injury, and/or disc degeneration.

The cell microsheets of the present invention are formed from a culture of cells. Cells used for formation of cell microsheets should preferably be animal cells, more preferably mammal cells, further preferably primate cells, most preferably human cells.

In one embodiment of the present invention, cells for formation of the cell microsheets may be derived from cartilage tissues. In detail, the cell microsheet of the present invention may be formed from a culture of cells derived from cartilage tissue. The cell microsheets of the present invention are more suitable for cartilage tissue repair.

The cells derived from cartilage tissue may be prepared, for example, through separation of cells contained in the cartilage tissue from the cartilage tissue. In detail, cells derived from the cartilage tissue may be prepared by treatment of the cartilage tissue with an enzyme to liberate cells in the cartilage tissue from the cartilage tissue and then centrifugal recovery of the liberated cells.

In the present invention, the cartilage tissue may be that of an animal with polydactyly or that of an animal with polymelia, which is an animal with extra fingers and/or toes. The animal may preferably be a mammal, more preferably a primate, most preferably a human. The cartilage tissue can be collected from tissues obtained, for example, during excision of the excess finger or toe. Such tissue may be, for example, a portion that does not appear white, but appears black in an x-ray photograph. The polydactyly may be of a distal, intermediate, or proximal phalanx type. The extra finger or toe may be any finger or toe, for example, pollex or digitus minimus. If the extra fingers or toes to be collected are wart-like and small, the entire collected subcutaneous tissue can be used. If the animal is a human, the age of the human should preferably be 5 years or younger, more preferably 3 years or younger, even more preferably 2 years or younger, even more preferably 1 year or younger, for more efficient cultivation.

Examples of the enzyme used in the enzyme treatment include collagenase, caseinase, clostripain, trypsin, hyaluronidase, elastase, pronase, and dispase. In preferred embodiments, these enzymes could be used in combination. A preferable example of enzyme combination is a combination of collagenase, caseinase, clostripain, and trypsin. Examples of enzyme preparations containing this combination include, but not limited to, collagenase type I, collagenase type II, collagenase type III, collagenase type IV, and collagenase type V (all available from Fujifilm Wako Pure Chemical Corporation). Another example of a preferred enzyme combination is a combination of collagenase with dispase or thermolysin. Examples of enzyme preparations containing this combination include, but are not limited to, Liberase (available from Roche Diagnostics K.K.). The enzyme treatment may be carried out stepwise with different enzymes, depending on the state of the tissue. For example, isolation may be performed by treatment with collagenase, caseinase, clostripain and then trypsin in this order. Conditions for enzyme treatment can be appropriately determined by those skilled in the art depending on the type of enzyme used and/or the condition of cartilage tissue. The enzyme treatment can be carried out at, for example, 30 to 50° C., preferably 33 to 45° C., 35 to 40° C. for, for example, 1 to 12 hours, preferably 2 to 5 hours. An excess high-temperature in the enzyme treatment arises problems such as cell denaturation, loss of living cells, decreased proliferative capacity, and inability to isolate can occur. An extra low temperature of the enzyme treatment causes insufficient enzyme activity for isolation of cells. Application of physical stimulation during enzyme treatment can achieve cell recovery with high efficiency.

The enzymatic reaction can be terminated by dilution of the enzyme through washing of the cell suspension after the articular cartilage is enzymatically treated. After termination of the enzymatic reaction, the cell suspension can be separated into a cell mass and a supernatant by centrifugation. For Liberase, the enzymatic reaction can be terminated by washing two or more times. The centrifugation can be performed under conditions where a greater number of cells of less than 25 μm, in particular 20 μm or less and 15 μm or more can be collected. In order to collect a greater number of such cells, the centrifugation can be performed, for example, at 1000 rpm or higher, 1500 rpm or higher, or 2000 rpm or higher, for, for example, 5 minutes or longer, 7 minutes or longer, or 10 minutes or longer.

The cells derived from the cartilage tissue may be prepared by a so-called outgrowth process. The outgrowth process may involve a step of chopping the collected cartilage tissue and a step of seeding the chopped cartilage tissue piece in a culture dish with a small amount of medium and culturing the cells. The cultivation produces proliferated cells from the cartilage tissue pieces. The generated cells are recovered by enzymatic treatment and centrifugation. The recovered cells can be used for production of the cell sheet pieces of the present invention.

The step of chopping the cartilage tissue into small pieces can be performed, for example, in a wet state. The step can be performed, for example, by placing pieces of tissue and a small amount of medium in a 50 ml centrifuge tube and chopping it with Metzenbaum Scissors, SuperCut Tungsten Carbide 18 cm Long Curve (World Precision Instruments). It is preferable to obtain as small a piece of cartilage tissue as possible. The medium for culturing the finely chopped cartilage tissue pieces can be appropriately selected by those skilled in the art. A preferred medium is DMEM/F12+20% FBS+antibiotic (hereinafter, also referred to as AB). After the adhesion of cells to the culture dish is confirmed after the start of cultivation, the medium can be preferably replaced with DMEM/F12+20% FBS+AB+ascorbic acid (hereinafter also referred to as AA). If the medium contains ascorbic acid from the beginning of the cultivation, ascorbic acid may inhibit the adhesion of cells to the culture dish. In addition, the cultivation may be carried out under general cultivation conditions, for example, in an incubator at 37° C. and 5% CO₂. The cultivation can be carried out until subconfluence. The enzyme preparation used in the recovery of cells produced in cultivation can include, for example, trypsin and EDTA. Centrifugation can be performed as described above.

In the present invention, the cartilage tissue-derived cells may preferably contain mesenchymal stem cells. The cells derived from the cartilage tissue may further contain cells contained in the cartilage tissue in addition to the mesenchymal stem cells. That is, in the present invention, the cartilage tissue-derived cells can be a group of different types of cells including mesenchymal stem cells. Examples of cells other than mesenchymal stem cells include, but are not limited to, chondrocytes and chondroblasts.

In another embodiment of the invention, the cells used to form the cell microsheet may be derived from stem cells. The stem cells may include pluripotent stem cells, such as iPS cells, embryonic stem cells, or somatic stem cells.

In this embodiment, the cell microsheet of the present invention may be prepared, for example, through differentiation by cultivation of pluripotent stem cells (particularly iPS cells) in a medium, and further cultivation of the chondrocytes or cartilage-like cells on the surface on which a temperature-responsive polymer is immobilized. The cell microsheet of the present invention can also be produced through, for example, seeding pluripotent stem cells (particularly iPS cells) on a cell cultureware on which a temperature-responsive polymer is immobilized, and culturing them by differentiating them into chondrocytes or cartilage-like cells.

The cell microsheet of the present invention can be produced by a method of the present invention described in Section 3.

Section 2. Syringe containing cell microsheets

The present invention also provides a syringe containing the cell microsheets. The features of the cell microsheets have been described in Section 1. The syringe of the present invention preferably contains the cell microsheets in a form of dispersion. The syringe containing the cell microsheets of the present invention is preferably used in medical treatment.

The syringe of the present invention includes an injection needle and a plunger. The injection needle may have any thickness that can be appropriately determined by a person skilled in the art. The thickness of the needle is discussed in Section 1.

Section 3. Production of cell microsheet

The present invention further provides a method of producing cell microsheets. The method involves cultivating cells on a surface of a cell cultureware provided with stimulus-responsive polymer immobilized thereon and having small divisions formed thereon to yield the cell microsheets. The cell microsheets produced by the method are preferably used for cartilage tissue repair. Accordingly, the method of the present invention may be a method of producing cell microsheets for cartilage tissue repair.

The cells cultivated in the method of the present invention may be derived from a cartilage tissue or a stem cell discussed in Section 1. For example, the source cells used for cultivation may be the above-mentioned cartilage tissue-derived cells, and the cartilage tissue-derived cells are prepared by cultivation of the cells in the cartilage tissue in DMEM/F12 containing FBS for at least two days.

In the method of the present invention, cells may be cultivated on a surface of a cell cultureware where the stimulus-responsive polymer is immobilized on the surface. Cultivation of cells in a medium containing a cell cultureware having a surface on which a stimulus-responsive polymer is immobilized enables the cells to be detached from the cell cultureware after cultivation without damage of the culture (i.e. cell microsheets) on the surface. The stimulus-responsive polymer may be, for example, a temperature-responsive polymer, a pH-responsive polymer, or a photoresponsive polymer, and more specifically a polymer having variable properties, such as hydration ability, by temperature stimulus (for example, temperature change), or pH stimulus (for example, pH change), or light stimulus (for example, light irradiation). The changes in properties may be, for example, changes in properties that promote detachment of the culture from the cell cultureware.

In one embodiment of the invention, the stimulus-responsive polymer is a temperature-responsive polymer that has an upper critical solution temperature or a lower critical solution temperature. After the cells are cultivated in a medium including a cell cultureware having a surface on which the temperature-responsive polymer is immobilized, the temperature of the medium is set to be above the upper critical solution temperature of the temperature-responsive polymer having an upper critical solution temperature or below the lower critical solution temperature of the temperature-responsive polymer of the temperature-responsive polymer having a lower critical solution temperature. The surface thereby changes from hydrophobic to hydrophilic, and the cell microsheet can be readily detached from the cell cultureware. The cultivation in the method of the present invention may preferably be two-dimensional cultivation (also referred to as planar cultivation).

The cell microsheet can be detached from the cell cultureware after cultivation without treatment using a proteolytic enzyme, such as dispase and trypsin. By utilizing the characteristics of the temperature-responsive polymer and changing the temperature of the medium, the cell microsheet can be detached from the cell cultureware. The cell microsheet produced by the method of the present invention thus has an advantage in that it can be detached from the cell cultureware without enzymatic damage.

Treatment with proteolytic enzymes leads to the degradation of cell-to-cell desmosome structures and cell-to-cultureware basement membrane-like proteins, which can result in separation of cells in cell culture into individual cells. In contrast, the cell microsheet prepared by the method of the present invention can be detached from the cell cultureware by changing the temperature of the medium without treatment using a proteolytic enzyme. As a result, the desmosome structure can be retained and defects in cell microsheet can be reduced. Furthermore, the cell microsheet prepared by the method of the present invention can be detached from the cell cultureware by changing the temperature of the medium, without enzymatic damage of the basement membrane-like protein. The cell microsheet thus can satisfactorily effects on the affected tissue, resulting in efficient treatment.

Although dispase, which is a proteolytic enzyme, is known to be able to detach a cell sheet while retaining 10 to 60% of the desmosome structure, it almost destroys the basement membrane-like protein; hence, the cell culture has weak strength. The cell microsheet produced by the method of the present invention can be detached from the cell cultureware while 80% or more of the desmosome structure and the basement membrane-like protein remain.

The upper critical solution temperature of the thermosensitive polymer having the upper critical solution temperature and the lower critical solution temperature of the thermosensitive polymer having the lower critical solution temperature used in the present invention both range from preferably 0 to 80° C., more preferably 20 to 50° C., most preferably 25 to 45° C. If the upper critical solution temperature or the lower critical solution temperature exceeds the upper limit of the range, cells can die. If the upper critical solution temperature or the lower critical solution temperature falls below the lower limit of the range, cell proliferation can slow or cells can die.

In the method of the present invention, the temperature-responsive polymer may be either a homopolymer or a copolymer. The polymer may be, for example, a homopolymer of a (meth)acrylamide compound, an N-(or N,N-di-)alkyl-substituted (meth)acrylamide derivative, or a vinyl ether derivative, or a copolymer of these monomers.

Examples of the (meth)acrylamide compounds include acrylamide and methacrylamide.

Examples of the N-alkyl-substituted (meth)acrylamide derivative include N-ethylacrylamide (lower critical solution temperature of the homopolymer: 72° C.), N-n-propylacrylamide (ditto: 21° C.), N-n-propylmethacrylamide (ditto: 27° C.), N-isopropylacrylamide (ditto: 32° C.), N-isopropylmethacrylamide (ditto: 43° C.), N-cyclopropylacrylamide (ditto: 45° C.), N-cyclopropylmethacrylamide (ditto: 60° C.), N-ethoxyethylacrylamide (ditto: about 35° C.), N-ethoxyethylmethacrylamide (ditto: about 45° C.), N-tetrahydrofulfurylacrylamide (ditto: about 28° C.), and N-tetrahydrofulfurylmethacrylamide (ditto: about 35° C.).

Examples of the N,N-dialkyl-substituted (meth)acrylamide derivative include N,N-dimethyl(meth)acrylamide, N,N-ethylmethylacrylamide (lower critical solution temperature of the homopolymer: 56° C.) and N,N-diethylacrylamide (ditto: 32° C.).

The vinyl ether derivative is, for example, methyl vinyl ether (lower critical solution temperature of the homopolymer: 35° C.).

In the present invention, the temperature-responsive polymer may also be a copolymer with a monomer other than the above-mentioned monomer, a graft polymer, a copolymers, or a mixture of two or more polymers or copolymers. These (co)polymers may be cross-linked.

From among these polymers, a suitable temperature-responsive polymer with an upper or lower critical solution temperature suitable for cultivation and detachment in the present invention may be appropriately selected to regulate the interaction between the cell cultureware and the culture, or to adjust the hydrophobic or hydrophilic characteristics of the surface of the cell cultureware.

In preferred embodiments, the thermosensitive polymer is poly(N-isopropylacrylamide).

In the method of the present invention, the amount of the temperature-responsive polymer to be immobilized on the surface of the cell cultureware is preferably 0.3 to 5.0 μg/cm², more preferably 0.3 to 4.8 μg/cm². The immobilized temperature-responsive polymer present in an amount within this range can enhances the efficiency of cultivation. An amount outside of this range of the immobilized polymer may preclude or reduce the formation of the cell microsheets. Within this range of the immobilized polymer, the cell microsheets can be readily detached from the cell cultureware.

In the method of the present invention, the cell cultureware may have any shape. Examples of the shape include, but not limited to, dishes, multi-plates, flasks, and cell inserts.

The immobilization of the temperature-responsive polymer on the surface of the cell cultureware can be performed by, for example, a process described in Japanese Unexamined Patent Application Publication No. H2-211865. In detail, the immobilization can be performed by bonding the cell cultureware with the temperature-responsive polymer by a chemical reaction, a physical interaction, or a combination thereof.

The binding process by the chemical reaction involves, for example, electron beam irradiation (EB), γ-ray irradiation, ultraviolet irradiation, visible light irradiation, LED light irradiation, plasma treatment, or corona treatment. Alternatively, the temperature-responsive polymer may be immobilized on the cell cultureware by a commonly used organic reaction, such as a radical reaction, an anionic radical reaction, or a cationic radical reaction. Alternatively, the temperature responsive polymer may be a block copolymer having a structure consisting of a water-insoluble polymer segment and a temperature-responsive polymer segment. The temperature responsive polymer may be immobilized by physical adsorption or hydrophobic treatment.

The binding process by the physical interaction involves, for example, application of a mixture of a temperature responsive polymer and any medium onto the cell cultureware.

In the method of the present invention, cells are cultured on a surface having small divisions of the cell cultureware. Culturing the cells on the surface having such small divisions can readily yield cell microsheets. The small divisions may each have an area of preferably 20 mm² or less, more preferably 15 mm² or less, and even more preferably 10 mm² or less. An excessively small area may preclude formation of cell microsheets from a cell culture. Accordingly, the area should preferably be 0.02 mm² or more.

The cell cultureware having a surface on which the temperature-responsive polymer is immobilized and small divisions are formed are commercially available, for example, RepCell (CellSeed Inc.), which is a temperature-responsive culture dish provided with a grid wall on the culture surface

The medium used in the cultivation used in the method of the present invention may be one that can be used for cell cultivation, in particular for cultivation of mammalian cells, such as DMEM/F12 (Dulbecco's Modified Eagle Medium: Nutrient Mixture F-12). The medium may contain any additive factor. Examples of the additive factor include cell growth factors, hormones, binding proteins, cell adhesion factors, lipids, and other components.

Examples of the cell growth factor include, but not limited to, TGF-β, b-FGF, IGF, epidermal growth factors (EGFs), bone morphogenetic proteins (BMPs), fibroblast growth factor receptor 3 (FGFR-3), Frizzled-related proteins (FRZBs), CDMP-1, growth differentiation factor 5 (GDF-5), granulocyte colony stimulating factors (G-CSFs), leukemia inhibitory factors (LIFs), interleukins, platelet-derived growth factors (PDGFs), nerve growth factors (NGFs), transforming growth factors (TGFs) such as activing A, and Wnt family, in particular Wnt-3a (wingless-type MMTV integration site family, member 3A). Examples of the TGF family include TGF-β1, TGF-β2, and TGF-β3.

Examples of the hormone include, but not limited to, insulin, transferrin, dexamethasone, estradiol, prolactin, glucagon, thyroxine, growth hormone, follicle stimulating hormone (FSH), luteinizing hormone (LH), glucocorticoid, and prostaglandin.

Examples of the cell adhesion factor include, but are not limited to, collagen, collagen-like peptides, fibronectin, laminin, and vitronectin. Examples of the collagen-like peptide include a recombinant peptide in which the RGD sequence-containing region in collagen is linked. Examples of such recombinant peptides include cellnest (FUJIFILM Corporation).

Examples of the lipid include, but are not limited to, phospholipids and unsaturated fatty acids.

Examples of the other components that can be added to the medium include, but not limited to, ascorbic acid, serum, insulin-transferrin-sodium selenite (ITS), transferrin, sodium selenite, pyruvic acid, proline, albumin, lipoproteins, and ceruloplasmin. Examples of the serum include, but not limited to, fetal bovine serum (FBS) and human serum. In the method of the present invention, the medium is preferably a medium containing FBS, in particular, DMEM/F12 containing FBS. The content of FBS for more efficient cultivation is preferably 1 to 30% by volume, preferably 10 to 30% by volume, more preferably 12 to 28% by volume, even more preferably 15-25% by volume, based on the total volume of the medium.

In another embodiment of the method of the present invention, the medium may be a serum-free medium. Since additive factors, such as ITS, can function as a replacement for serum, a medium including the additive factor enables the cultivation according to the method of the present invention in a serum-free medium. The use of serum-free medium can also avoid the risk accompanied by use of biological resources for administration to humans.

The medium used in the production of cell microsheets for cartilage tissue repair of the present invention contains ascorbic acid in an amount of usually 0.01 to 1 mg/mL, preferably 0.05 to 0.5 mg/mL, more preferably 0.07 to 0.3 mg/mL relative to the volume of the medium volume, for superior cell proliferation. Ascorbic acid can promote the production of articular cartilage-specific substrates from cultured cells and/or the expression more suitable for cartilage tissue repair of phenotype of the resulting cell microsheets. Ascorbic acid can accordingly contribute to the regeneration of damaged sites of articular cartilage by hyaline cartilage. An extra content of ascorbic acid may inhibit the adhesion of the cultured cells to the cell cultureware. A significantly low content of the ascorbic acid may result in insufficient effect.

In a preferred embodiment, the cell microsheets of the present invention can be produced through cultivation of cells in a medium, preferably in a medium containing FBS and/or ascorbic acid, for example, in DMEM/F12 containing FBS and/or ascorbic acid.

In the method of the present invention, the cultivation period of the cells on the cell cultureware may be appropriately selected depending on the state of the culture, for example, a phenotypic state. The cultivation period is, for example, 10 to 20 days, preferably 11 to 18 days, more preferably 12 to 16 days. Cell microsheets produced during such a cultivation period are more suitable for cartilage repair.

The characteristics of the cell microsheets prepared by the method of the present invention are as described in Section 1. The cells cultured in the method of the present invention and the method for preparing the cells are also described in Section 1.

Section 4. Use of cell microsheet

The present invention also provides a method of administering cell microsheets to an animal by injection. The cell microsheets are described in Section 1. In the method of the present invention, the cell microsheets may be administered to the animal with a syringe containing the cell microsheet described in Section 2. The animal may be more preferably a mammal, even more preferably a primate, and particularly preferably a human.

The method of the present invention can be applied to repairing cartilage tissue. The method of the present invention can administer the cell microsheets by injection and thus is less invasive treatment. Examples of cartilage tissue repair in the methods of the invention includes, but not limited to, treatment of inflamed and/or damaged cartilage tissue, reinforcement of cartilage tissue, compensation of defective parts of cartilage tissue, and regeneration of cartilage tissue. The present invention may also be applied to preventing a disease related to cartilage tissue. In the method of the invention, cell microsheets can be administered, for example, by injection into diseased cartilage or bone tissue. Examples of diseases to which the methods of the invention is applicable include, but are not limited to, arthritis, arthropathy, cartilage injury, osteochondral injury, meniscus injury, and/or disc degeneration.

### EXAMPLES

The present invention will now be described in more detail by way of examples that should not be construed to limit the present invention.

### Example 1

(Preparation of cell microsheet)

A temperature-responsive culture dish (RepCell, CellSeed Inc.) having a cultivation surface provided with a 3 mm by 3 mm grid wall and a temperature-responsive culture insert (UpCell® insert, CellSeed Inc.) were provided. Chondrocytes isolated from cartilage tissue in patients with polydactyly was seeded at a density of 1×10⁴ cells/cm² on each of the culture dish and the culture insert. The chondrocytes were cultivated for two weeks. After each cell cultureware was allowed to stand at room temperature for 30 minutes or more, inventive cellular microsheets (Group M) of example were collected from the temperature-responsive culture dish, and a comparative cell sheet (Group S) was collected from the temperature-responsive culture insert. The comparative cell sheet (Group S) is a conventional cell sheet used for articular cartilage treatment.

(Evaluation of cell microsheet)

Cell microsheets (Group M) and the cell sheet (Group S) were evaluated according to the following items:

Measurement of number and viability of cells

Measurement of secretion of humoral factor by ELISA (melanoma inhibitory activity (MIA) and TGF-β1)

Quantitative analysis of cartilage-related gene expression by qPCR (COL1A1, COL2A1, COL10A1, ACAN, SOX9, RUNX2, and MMP3)

Analysis of cell surface markers (CD29, CD31, CD44, CD45, CD73, CD81, CD90, and CD105)

Evaluation of tissue sections (HE, safranin O, and toluidine blue stain)

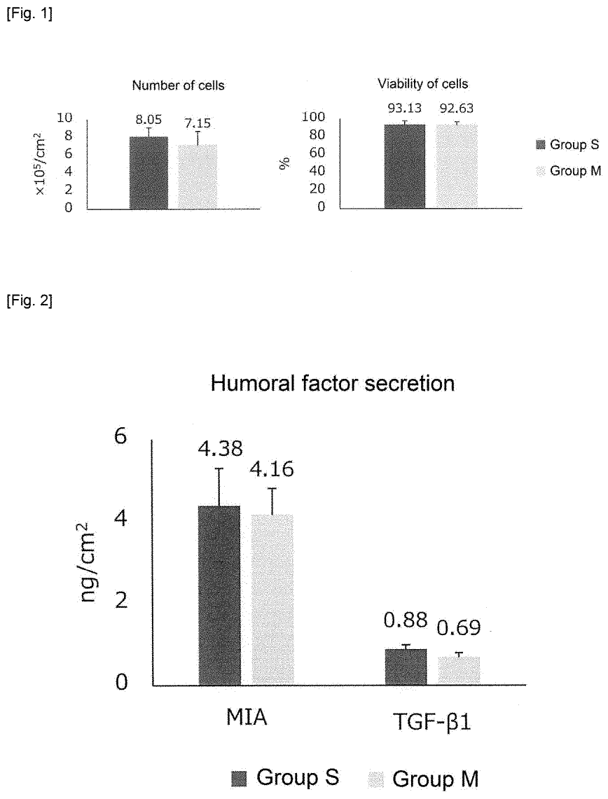

The results of these evaluations are represented in FIGS. 1 to 5.

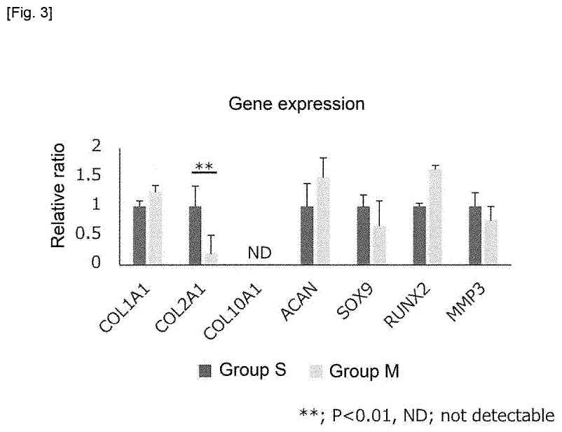

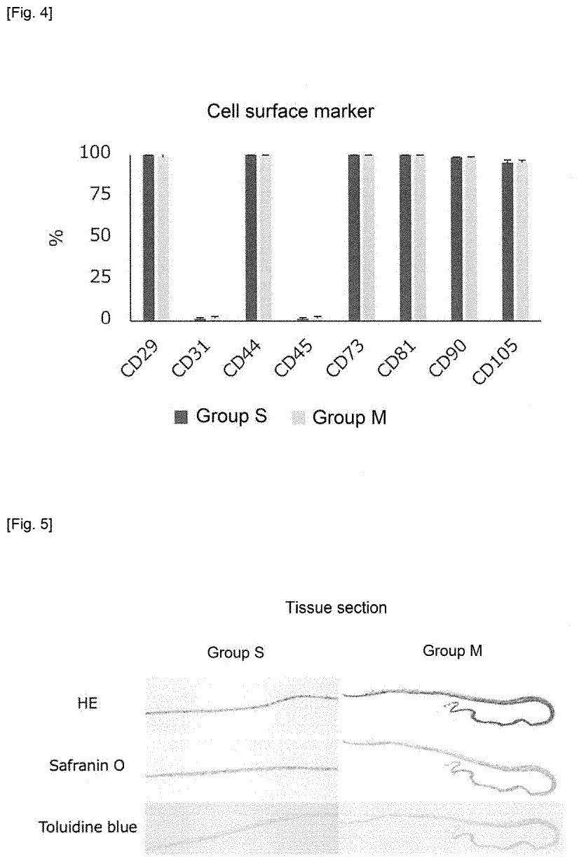

FIG. 1 demonstrate that no significant difference was observed in the number and viability of cells between the cellular microsheets (Group M) and the cell sheet (Group S). FIG. 2 demonstrate that no significant difference was observed in the amount of humoral factors secreted between the cell microsheets (Group M) and the cell sheet (Group S). FIG. 3 demonstrates that no significant difference is found in the expression levels of cartilage-related genes between the cell microsheets (Group M) and the cell sheet (Group S) except for COL2A1. In the cell microsheets (Group M), the expression level of COL2A1 was lower than that in the cell sheet (Group S). A possible cause is that the cell microsheets (Group M) were cultivated in the flat culture dish not in the insert. FIG. 4 demonstrates that no significant difference was observed in the cell surface markers between the cell microsheets (Group M) and the cell sheet (Group S). FIG. 5 demonstrates that stratification of chondrocytes was observed in both the cell microsheets (Group M) and the cell sheet (Group S), but both were poor in safranin O and toluidine blue stainability.

(Verification of effect of injection administration)

In order to verify the effect of injection administration on viability of cells, the cell microsheets (Group M) were divided into the following four groups, and a series of tests was conducted to compare the viability of cells immediately, 4 hours, and 24 hours after injection. Non-injection control group

G18 needle group

G23 needle group

Syringe group (syringe only without needle)

After cultivation for two weeks and allowing to stand at room temperature for 30 minutes or more, the cell microsheets (Group M) were collected from the temperature-responsive culture dish. The cell microsheets (Group M) were suspended in 3 mL of medium. For the injection-administered group, the suspension was gently injected into a 5 mL syringe and then passed through a G18 needle, a G23 needle, or a syringe. The viability of cells was measured by trypan blue assay at 0 hours (immediately after injection administration), 4 hours, and 24 hours after injection. The viability of cells of the cell sheet (Group S) was also measured 0 hours (immediately after detachment) and 24 hours after detachment from the temperature-responsive culture insert.

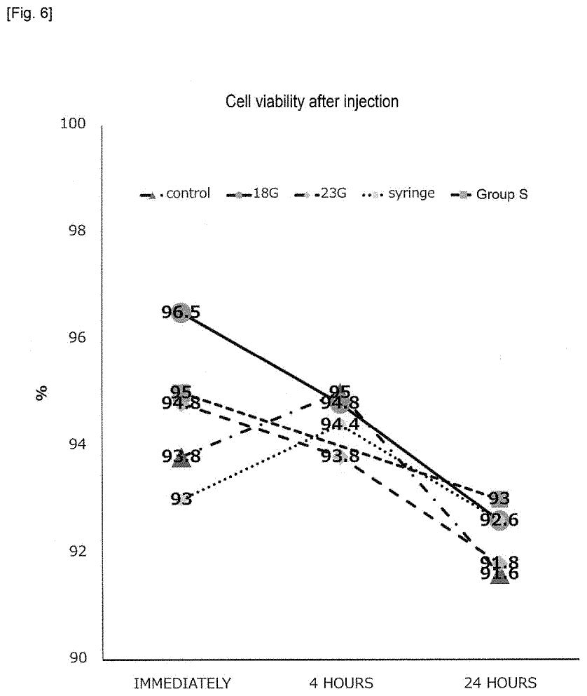

The results of the measurement of the viability of cells are shown in FIG. 6.

Cell microsheets (Group M) successfully passed through G18 and G23 injection needles in this test. FIG. 6 indicates that the viability of cells is 90% or more at each time in all the four groups. Although the viability of cells decreases with the passage of time, no significant difference is observed between the four groups at each time (immediately after administration: ρ=0.748, four hours later: ρ=0.987, after 24 hours: ρ=0.994). These results demonstrate that the cell microsheets of the present invention are barely affected by injection administration.

### Example 2

The cell microsheets of the present invention were compared with a conventional cell sheet to verify the therapeutic effect on the articular cartilage of the cell microsheets on a non-traumatic knee arthritis model.

Chondrocytes derived from polydactyly were seeded in a conventional temperature-responsive culture dish and a temperature-responsive culture dish (RepCell, CellSeed Inc.) having a culture surface provided with a grid wall (3 mm by 3 mm) to prepare inventive cell microsheets and a comparative cell sheet. The number and viability of cells, cell surface marker expression, COL2A1/COL1A1 expression ratio, and secretory volume of humoral factor were measured for comparison of sheet characteristics.

Monoiodoacetic acid (MIA) (0.2 mg/30 μL) was intraarticularly administered to the right knees of 18 nude rats F344/NJcl-rnu/rnu (aged 8 weeks) to prepare arthritis models, and saline (30 μL) was intraarticularly administered to the right knees of six nude rats as a control group.

The 18 arthritis models were distributed at random to a non-transplanted group after MIA administration (Group A), a cell sheet transplanted group after MIA administration (Group B), and a cell microsheet transplanted group after MIA administration (Group C) (n=6 for each). Four weeks later from MIA administration, the cell sheet was transplanted into the right knees in Group B, while suspension of cell microsheets in saline (30 μL) was injected it into the right knee joints with a G23 needle in Group C. Histological evaluation (OARSI score) was performed eight weeks after MIA administration.

The results demonstrates that no significant difference is found in the sheet characteristic evaluation items between the cell sheet and the cell microsheets. No significant difference is also found in COL2A1/COL1A1 expression ratio, melanoma inhibitory activity, and secreted amounts of TGF-β1, ESM1, MCP-1, DKK-1, MMP-13, and MMP-3, which are used for the evaluation of the effectiveness of the sheet.

The OARSI score on the femur side was 11.0±3.0 in Group A, 3.0±2.0 in Group B, 3.2±1.8 in Group C. The results demonstrate a significant improvement between Groups A and B and Groups A and C (P <0.01), and no significant difference between Groups B and C.

The results indicates that the sheet characteristics of the conventional cell sheet and the cell microsheets of the present invention are substantially the same as the conventional cell sheet, and the cell microsheets of the present invention that can be transplanted to be less invasive and have the same therapeutic effect on articular cartilage as the conventional cell sheet.

What is claimed is:

1. A cell microsheet that is formed from a culture of cells and is capable of passing through an injection needle.

2. The cell microsheet set forth in claim 1, wherein the injection needle is an 18G or thinner injection needle.

3. The cell microsheet set forth in claim 1, having an area of 20 mm² or less.

4. The cell microsheet set forth in claim 1, wherein the cell microsheet is usable for cartilage tissue repair.

5. The cell microsheet set forth in claim 1, wherein the cells are derived from cartilage tissue.

6. The cell microsheet set forth in claim 5, where the cartilage tissue is of an animal with polydactyly.

7. The cell microsheet set forth in claim 1, wherein the cells are derived from stem cells.

8. The cell microsheet set forth in claim 7, wherein the stem cells include pluripotent stem cells, embryonic stem cells, or somatic stem cells.

9. The cell microsheet set forth in claim 7, wherein the stem cells comprise iPS cells.

10. A syringe containing the cell microsheet set forth in claim 1.

11. A method of producing cell microsheets formed from a culture of cells, comprising cultivating the cells on a surface of a cell cultureware to yield the cell microsheets, a stimulus-responsive polymer being immobilized on the surface of the cell cultureware, the surface having small divisions.

12. The method set forth in claim 11, wherein the small divisions each have an area of 20 mm² or less.

13. A method of administering the cell microsheet set forth in claim 1 to an animal by injection.
